(19) United States
(12) Patent Application Publication
(10) Pub. No.: US 2026/0098859 A1
(43) Pub. Date: Apr. 9, 2026

(54) REAGENT SYSTEM FOR ACRIDINIUM ESTER CHEMILUMINESCENCE AND METHOD OF DETECTING TARGET ANTIGEN USING THE SAME

(71) Applicant: Ji Hoon LEE, Gaithersburg, MD (US)

(72) Inventor: Ji Hoon LEE, Gaithersburg, MD (US)

(21) Appl. No.: 18/907,416

(22) Filed: Oct. 4, 2024

(51) Int. Cl. G01N 33/53

(52) U.S. Cl. CPC G01N 33/5306; G01N 2470/04

(57) ABSTRACT

Provided is a reagent system for acridinium ester chemiluminescence, and a method of detecting a target antigen using the same. The reagent system for eliciting an acridinium ester chemiluminescence includes: a wash buffer including a first surfactant in one or more of a phosphate-buffered saline (PBS) and a tris-buffered saline (TBS); a first reagent including hydrogen peroxide and an acid, and optionally a first alkali metal salt; and a second reagent including a base and a second surfactant, and optionally a second alkali metal salt, where the first surfactant and the second surfactant are independently one or more of a cationic surfactant, a neutral surfactant and an anionic surfactant, where the acid having an acid dissociation constant (pKa) of −1.0 to 5.0, and where the base comprises one or more of sodium hydroxide (NaOH), potassium hydroxide (KOH), ammonium hydroxide, tetra-n-ethylammonium hydroxide (TEA), tetra-n-propylammonium hydroxide (TPA), and tetra-n-butylammonium hydroxide (TBA).

### TECHNICAL FIELD

The present disclosure pertains to advancements in biochemical diagnostics, introducing an innovative chemiluminescence detection system for acridinium ester-labeled immunocomplexes, featuring a novel formulation of pre-activating and activating reagents with graphene oxide and specific surfactants, and delineating a method for enhanced target antigen detection.

### BACKGROUND

Details in the background section do not constitute the related art but are given only as background information concerning the subject matter of the present disclosure.

In traditional chemiluminescence detection systems designed for acridinium ester-labeled immunocomplexes, challenges such as elevated background noise and the requirement for high reagent concentrations complicate the assay procedures, adversely affecting both sensitivity and specificity. Such systems often require substantial amounts of hydrogen peroxide (H₂O₂) and potent acids in the first reagent, leading to unwanted background signals that can obscure accurate analyte quantification. Moreover, the use of these higher concentrations of chemicals not only poses potential safety hazards, but also escalates the environmental burden due to chemical disposal.

Recently, by the inventor of the present disclosure, advancements have highlighted the introduction of graphene oxide (GO) into a pre-activating reagent, characterized by a lower concentration of H₂O₂ and a milder acidic compound, effectively addressing the limitations faced by conventional chemiluminescence detection systems. The addition of GO not only improves the dispersion and stability of acridinium ester-labeled immunocomplexes but also acts as an oxidizing mediator, thereby enhancing the chemiluminescence reaction with reduced H₂O₂ levels. Furthermore, the composition of the second reagent is finely tuned to efficiently initiate chemiluminescence without contributing to the background noise. The strategic adjustment of the reaction mixture's pH, through the careful selection of the base and surfactant type and concentration, significantly enhances the detection system's sensitivity. However, the effectiveness of the chemiluminescence detection system, which relies on a combination of pre-activating solution containing graphene oxide (GO) and activating solutions, faced certain limitations.

The background description provided herein is for the purpose of generally presenting context of the disclosure. Unless otherwise indicated herein, the materials described in this section are not prior art to the claims in this application and are not admitted to be prior art, or suggestions of the prior art, by inclusion in this section.

### SUMMARY

The present disclosure has been made to solve the above-mentioned problems and other technical problems that have yet to be resolved.

An object of the present disclosure is to provide a reagent system having one wash buffer and two distinct reagents that facilitate a background-free analytical system for evaluating acridinium ester-labeled immunocomplexes from antigen-antibody interactions in immunoassays.

Another object of the present disclosure is to provide a method for detecting target antigens using the wash buffer and the first/second reagents that offer environmental benefits, cost efficiency, a low signal-to-background ratio, enhanced sensitivity, and rapid performance.

To fulfill the stated objective, an embodiment of the present disclosure provides a reagent system for eliciting an acridinium ester chemiluminescence, including: a wash buffer including a first surfactant in one or a combination of a phosphate-buffered saline (PBS) and a tris-buffered saline (TBS); a first reagent including hydrogen peroxide and an acid, and optionally a first alkali metal salt; and a second reagent including a base and a second surfactant, and optionally a second alkali metal salt, wherein the first surfactant and the second surfactant are independently one or more of a cationic surfactant, a neutral surfactant and an anionic surfactant, wherein the acid has an acid dissociation constant (pKa) of −1.0 to 5.0, and wherein the base comprises one or more of sodium hydroxide (NₐOH), potassium hydroxide (KOH), ammonium hydroxide, tetra-n-ethylammonium hydroxide (TEA), tetra-n-propylammonium hydroxide (TPA), and tetra-n-butylammonium hydroxide (TBA).

The first surfactant may be present in the wash buffer at a concentration of 0.05 to 0.3%. The first reagent may further comprise a graphene-based material selected from the group consisting of graphene, graphene oxide (GO), and reduced graphene oxide (rGO).

The acid having acid dissociation constant (pKa) of −1.0 to 5.0 may comprise one or more selected from the group consisting of acetic acid, citric acid, formic acid, nitric acid (HNO₃), oxalic acid, ascorbic acid, salicylic acid, hydrogen chloride, tartaric acid, lactic acid.

The cationic surfactant may comprise cetyltrimethylammonium chloride (CTAC) or cetyltrimethylammonium bromide (CTAB).

The neutral surfactant may be one or more polyethylene glycol tert-octylphenyl ether derivatives and/or polyoxyethylenesorbitan monopalmitate derivatives selected from the group consisting of Triton X-45, Triton X-100, Triton X-102, Triton X-114, Triton X-165, Triton X-305, Triton X-405, Triton X-705, Tween 20, Tween 40, Tween 60, Tween 80.

The anionic surfactant may comprise ammonium dodecyl sulfate (ADS) or sodium dodecyl sulfate (SDS).

The graphene-based material may be present in the first reagent at a concentration of more than 0 to 10 μg/ml.

The hydrogen peroxide may be present in the first reagent at a concentration of 2.5 to 50 mM, and wherein the second surfactant may comprise cetyltrimethylammonium chloride (CTAC) or cetyltrimethylammonium bromide (CTAB).

The hydrogen peroxide may be present in the first reagent at a concentration of 10 to 400 mM, and wherein the second surfactant comprises Triton X-100 or Tween 20.

The acid may be present in the first reagent at a concentration of 1 to 50 mM.

The second surfactant may be present in the second reagent at a concentration of 10 to 50 mM.

The base may be present in the second reagent at a concentration of 25 to 150 mM, and wherein the second surfactant may comprise cetyltrimethylammonium chloride (CTAC) or cetyltrimethylammonium bromide (CTAB).

The base may be present in the second reagent at a concentration of 100 to 400 mM, and wherein the second surfactant may comprise Triton X-100 or Tween 20.

The wash buffer may further comprise a preservative.

The preservative may comprise one or more selected from the group consisting of sodium azide, 5-bromo-5-nitro-1,3-dioxane, and a mixture of 5-chloro-2-methyl-4-isothiazolin-3-one with 2-methyl-4-isothiazolin-3-one.

Another embodiment of the present disclosure provides a method of detecting a target antigen in a sample, including: performing an immunoreaction between a target antigen in a sample and a detection antibody conjugated with acridinium ester for sandwich immunoassays or an artificial antigen, which is compatible with the target antigen, conjugated with acridinium ester for competitive immunoassays to form an acridinium ester-labeled immunocomplex; mixing a wash buffer including a first surfactant in one or more of a phosphate-buffered saline (PBS) and a tris-buffered saline (TBS) with the acridinium ester-labeled immunocomplex to form micelles around the acridinium ester-labeled immunocomplex; dispersing the acridinium ester-labeled immunocomplex within the micelles in a first reagent comprising hydrogen peroxide and an acid, and optionally a first alkali metal salt; adding a second reagent including a base and a second surfactant, and optionally a second alkali metal salt, into the dispersed acridinium ester-labeled immunocomplex to emit a chemiluminescence signal; and measuring an intensity of the chemiluminescence signal, wherein the first surfactant and the second surfactant are independently one or more of a cationic surfactant, a neutral surfactant and an anionic surfactant, wherein the acid has an acid dissociation constant (pKa) of −1.0 to 5.0, and wherein the base comprises one or more of sodium hydroxide (NaOH), potassium hydroxide (KOH), ammonium hydroxide, tetra-n-ethylammonium hydroxide (TEA), tetra-n-propylammonium hydroxide (TPA), and tetra-n-butylammonium hydroxide (TBA).

The dispersing step may be performed in a reaction vessel, wherein the reaction vessel may comprise the wash buffer in an amount of 50 μl or less and the acridinium ester-labeled immunocomplex.

The cationic surfactant may comprise cetyltrimethylammonium chloride (CTAC) or cetyltrimethylammonium bromide (CTAB).

The neutral surfactant may be one or more polyethylene glycol tert-octylphenyl ether derivatives and/or polyoxyethylenesorbitan monopalmitate derivatives selected from the group consisting of Triton X-45, Triton X-100, Triton X-114, Triton X-165, Triton X-305, Triton X-405, Triton X-705, Tween 20, Tween 40, Tween 60, and Tween 80.

The anionic surfactant may comprise ammonium dodecyl sulfate (ADS) or sodium dodecyl sulfate (SDS).

The first surfactant may be present in the wash buffer at a concentration of 0.05 to 0.3%.

The graphene-based material may be present in the first reagent at a concentration of 0.25 to 4 μg/ml.

The hydrogen peroxide may be present in the first reagent at a concentration of 2.5 to 40 mM, and wherein the second surfactant may comprise cetyltrimethylammonium chloride (CTAC) or cetyltrimethylammonium bromide (CTAB).

The hydrogen peroxide may be present in the first reagent at a concentration of 50 to 300 mM, and wherein the second surfactant may comprise Triton X-100 or Tween 20.

The acid may be present in the first reagent at a concentration of 1 to 50 mM.

The second surfactant may be present in the second reagent at a concentration of 10 to 50 mM.

The base may be present in the second reagent at a concentration of 25 to 150 mM, and wherein the second surfactant may comprise cetyltrimethylammonium chloride (CTAC) or cetyltrimethylammonium bromide (CTAB).

The base may be present in the second reagent at a concentration of 100 to 400 mM, and wherein the second surfactant may comprise Triton X-100 or Tween 20.

The wash buffer may further comprise a preservative.

The preservative may comprise one or more selected from the group consisting of sodium azide, 5-bromo-5-nitro-1,3-dioxane, and a mixture of 5-chloro-2-methyl-4-isothiazolin-3-one with 2-methyl-4-isothiazolin-3-one.

The sample may be selected from the group consisting of serum, plasma, whole blood, stool, cerebrospinal fluid, synovial fluid, tissue, nasal swabs, and urine.

The sample may be selected from the group consisting of drinking water, tap water, a vegetable, a fruit, a meat, and a contaminated material.

The target antigen may be an antigen derived from bacteria, cells, foodborne pathogens, peptides, proteins, haptens, or viruses.

The target antigen may comprise a small molecule antigen, a non-protein antigen, a cyclic antigen or a single epitope antigen.

The first reagent may further comprise a graphene-based material selected from the group consisting of graphene, graphene oxide (GO), and reduced graphene oxide (rGO).

The effects of the exemplary embodiments of the present disclosure are not limited to those mentioned above, and other effects not mentioned may be clearly derived and understood by one having ordinary skill in the art to which the exemplary embodiments of the present disclosure belong from the following description. In other words, unintended effects of practicing the exemplary embodiments of the present disclosure may also be derived from the exemplary embodiments of the present disclosure by one having ordinary skill in the art.

### BRIEF DESCRIPTION OF THE DRAWINGS

The accompanying drawings illustrate an embodiment of the present disclosure and together with the foregoing disclosure, serve to provide further understanding of the technical features of the present disclosure, and thus, the present disclosure is not construed as being limited to the drawing.

FIG. 1 is a schematic illustration showing chemiluminescence reaction procedure to quantify acridinium ester-labeled immunocomplexes for an immunoassay.

FIG. 2 is a graph showing the effect of surfactants added to TBS in acridinium ester chemiluminescence reaction. The concentration of individual surfactant in TBS is 0.1%. The total concentration of the combined surfactants in TBS was 0.1%. The first reagent contains 1 μg/ml of GO, 15 mM H₂O₂, and 12 mM citric acid. The second reagent contains 30 mM CTAC in 100 mM KOH.

FIG. 3 is a graph showing the volume effect of TBST in acridinium ester chemiluminescence immunoassays. The first reagent contains 1 μg/ml of GO, 15 mM H₂O₂, and 12 mM citric acid. The second reagent contains 30 mM CTAC in 100 mM KOH.

FIG. 4 is a graph showing the incubation time between acridinium ester-labeled immunocomplexes in TBSS and the first reagent before adding the second reagent to generate chemiluminescence. The first reagent contains 1 μg/ml of GO, 15 mM H₂O₂, and 12 mM citric acid. The second reagent contains 30 mM CTAC in 100 mM KOH.

FIG. 5 is a graph showing the stability of acridinium ester-labeled immunocomplexes in TBS and wash buffer such as TBST and TBSS at room temperature. The first reagent contains 1 μg/ml of GO, 15 mM H₂O₂, and 12 mM citric acid. The second reagent contains 30 mM CTAC in 100 mM KOH.

FIG. 6 is a graph showing the effect of GO concentration on the acridinium ester chemiluminescence reaction of acridinium ester immunocomplexes in TBSS. The first reagent contains GO, 15 mM H₂O₂, and 12 mM citric acid. The second reagent contains 30 mM CTAC in 100 mM KOH.

FIG. 7 is a graph showing the effect of H₂O₂ concentration on the acridinium ester chemiluminescence reaction operated with CTAC in KOH as the second reagent. The first reagent contains 1 g/ml GO, H₂O₂, and 12 mM citric acid. The second reagent contains 30 mM CTAC in 100 mM KOH.

FIG. 8 is a graph showing the effect of citric acid concentration on the acridinium ester chemiluminescence reaction operated with CTAC in KOH as the second reagent. The first reagent contains 1 μg/ml of GO, 15 mM H₂O₂, and citric acid. The second reagent contains 30 mM CTAC in 100 mM KOH.

FIG. 9 is a graph showing the effect of CTAC concentration on the acridinium ester chemiluminescence reaction. The first reagent contains 1 g/ml of GO, 15 mM H₂O₂, and 12 mM citric acid. The second reagent contains CTAC in 100 mM KOH.

FIG. 10 is a graph showing the effect of NaOH concentration on the acridinium ester chemiluminescence reaction for quantifying acridinium ester-labeled immunocomplexes in TBSS. The first reagent contains 1 μg/ml of GO, 15 mM H₂0₂, and 12 mM citric acid. The second reagent contains 30 mM CTAC in NaOH.

FIG. 11A is a graph showing the calibration curves of acridinium ester-labeled immunocomplexes in TBS and TBSS.

FIG. 11B is a graph showing CL_{TBSS}/CL_{TBS} determined with calibration curves shown in FIG. 11A.

FIG. 11C is a graph showing the comparison of an empirical calibration curve, derived from RCL measurements at high concentrations of acridinium ester-labeled immunocomplexes in TBS, with a theoretical calibration curve, computed using the first-order equation from the calibration curve data in TBS as depicted in FIG. 11A.

FIG. 11D is a graph showing the comparison of an empirical calibration curve, derived from RCL measurements at high concentrations of acridinium ester-labeled immunocomplexes in TBSS, with a theoretical calibration curve, computed using the first-order equation from the calibration curve data in TBSS as depicted in FIG. 11A. The first reagent contains 1 μg/ml GO, 10 mM H₂O₂, and 24 mM citric acid. The second reagent contains 50 mM KOH and 30 mM CTAC.

FIG. 12 is a graph showing the GO concentration effect in TBS and TBST for acridinium ester chemiluminescence in the presence of Triton X-100. The first reagent contains GO, 100 mM H₂O₂, and 12 mM citric acid. The second reagent contains 200 mM KOH and 1.5% Triton X-100.

FIG. 13 is a graph showing the effect of citric acid concentration on the acridinium ester chemiluminescence triggered by the second reagent containing Triton X-100. The first reagent contains GO, 100 mM H₂O₂, and citric acid. The second reagent contains 200 mM KOH and 1.5% TritonX-100.

FIG. 14 is a graph showing the effect of different acidic compounds on the acridinium ester chemiluminescence reaction in the presence of Triton X-100. The first reagent contains 1 μg/ml GO, 100 mM H₂O₂, and 12 mM acidic compound. The second reagent contains 200 mM KOH and 1.5% TritonX-100.

FIG. 15 is a graph showing the effect of H₂O₂ concentration on the acridinium ester chemiluminescence triggered by the second reagent containing Triton X-100. The first reagent contains 1 μg/ml GO, H₂O₂, and 12 mM acidic compound. The second reagent contains 200 mM KOH and 1.5% TritonX-100.

FIG. 16 is a graph showing the determination of Triton X-100 concentration in the second reagent for acridinium ester chemiluminescence reaction for quantifying acridinium ester-labeled immunocomplexes in wash buffer such as TBST, TBSS, TBSC. The first reagent contains 1 μg/ml GO, 100 mM H₂O₂, and 12 mM acidic compound. The second reagent contains 200 mM KOH and TritonX-100.

FIG. 17 is a graph showing the effect of NaOH concentration on the acridinium ester chemiluminescence reaction in the presence of Triton X-100. The first reagent contains 1 μg/ml GO, 100 mM H₂O₂, and 12 mM acidic compound. The second reagent contains NaOH and TritonX-100.

FIG. 18A shows the calibration curves of acridinium ester-labeled immunocomplexes in TBS and TBSS.

FIG. 18B is a graph showing CL_{TBSS}/CL_{TBS} determined with calibration curves shown in FIG. 18A.

FIG. 18C is a graph showing the comparison of an empirical calibration curve, derived from RCL measurements at high concentrations of acridinium ester-labeled immunocomplexes in TBS, with a theoretical calibration curve, computed using the first-order equation from the calibration curve data in TBS as depicted in FIG. 18A.

FIG. 18D is a graph showing the comparison of an empirical calibration curve, derived from RCL measurements at high concentrations of acridinium ester-labeled immunocomplexes in TBSS, with a theoretical calibration curve, computed using the first-order equation from the calibration curve data in TBSS as depicted in FIG. 18A. The first reagent contains 1 μg/ml GO, 100 mM H₂O₂, and 10 mM citric acid. The second reagent contains 200 mM NaOH and 1.5% Triton X-100.

### DETAILED DESCRIPTION

Hereinafter, the present disclosure will be described in more detail for a better understanding of the invention.

While the present disclosure is open to various modifications and alternative embodiments, specific embodiments thereof will be described and illustrated by way of example in the accompanying drawings. However, this is not purported to limit the present disclosure to a specific disclosed form, but it shall be understood to include all modifications, equivalents and substitutes within the idea and the technological scope of the present disclosure.

In this application, it should be understood that terms such as “include” or “have” are intended to indicate that there is a feature, number, step, operation, component, part, or a combination thereof described on the specification, and they do not exclude in advance the possibility of the presence or addition of one or more other features or numbers, steps, operations, components, parts or combinations thereof.

Hereinafter, the present disclosure will be described in detail.

The present disclosure can significantly enhance the chemiluminescence detection system through the integration of a sample containing acridinium ester-labeled immunocomplexes, which arise from immunoreactions, with a first reagent designed to pre-activate the immunocomplexes for chemiluminescence emission, and a second reagent formulated to initiate the chemiluminescence response. The introduction of the first surfactant into the sample plays a multifaceted role. It not only disperses the immunocomplexes, improving their stability through a synergistic interaction with graphene-based material, e.g., graphene oxide (GO), in the first (or pre-activating) reagent but also amplifies chemiluminescence intensity via cooperative effects between the first surfactant in the sample and a different surfactant type (second surfactant) in the second (or activating) reagent. The choice of first surfactant in the sample-whether anionic surfactants like ammonium dodecyl sulfate (ADS) and sodium dodecyl sulfate (SDS), cationic surfactants such as cetyltrimethylammonium bromide (CTAB) and cetyltrimethylammonium chloride (CTAC), or neutral surfactants including Triton X-100, which is a derivative of polyethylene glycol tert-octylphenyl ether, and Tween 20 which is a derivative of polyoxyethylenesorbitan monopalmitate,—is tailored to maximize the chemiluminescence detection system's sensitivity for acridinium ester chemiluminescence immunoassays, offering an advanced solution to enhance detection capabilities. The present disclosure allows for the use of various surfactant classes, including cationic, neutral, and anionic surfactants, enhancing the versatility and adaptability of the detection method.

This innovation addresses the drawbacks of traditional reagents by introducing a safer, eco-friendly, and highly sensitive method for detecting acridinium ester-labeled immunocomplexes. It is apt for diverse biochemical diagnostic applications, enhancing accuracy and minimizing environmental impact.

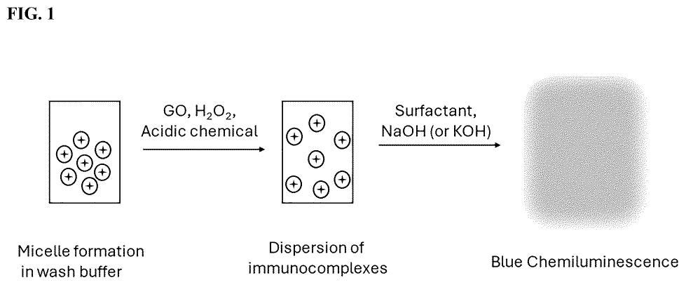

The reagent system according to the present disclosure can significantly enhance the chemiluminescence intensity and broaden the dynamic range of the linear calibration curve for quantifying acridinium ester-labeled immunocomplexes. These complexes arise from the interaction between a target antigen and an antibody that has been conjugated with acridinium ester, crucial in chemiluminescence immunoassays. The aspects of the present disclosure are depicted in FIG. 1, accompanied by a detailed procedural description for detecting acridinium ester chemiluminescence.
(1) Formation of micelles through the interaction of acridinium ester-labeled immunocomplexes with a wash buffer, such as PBS or TBS, that contains trace amounts of a first surfactant. The first surfactant can be anionic (e.g., ADS, SDS), cationic (e.g., CTAB, CTAC), or neutral (e.g., Triton X-100, Tween 20), facilitating the dispersion of immunocomplexes.
(2) Further dispersion of the immunocomplexes under the micelle condition following the incorporation of the first reagent, which includes a graphene-based material (e.g., graphene oxide (GO)), hydrogen peroxide (H₂O₂ ), and an acid. The acid can be acetic acid, ascorbic acid, citric acid, formic acid, nitric acid, or oxalic acid, contributing to the optimal conditioning of the reaction environment.
(3) Induction of intense chemiluminescence upon the addition of the second reagent, which comprises sodium hydroxide (NaOH) or potassium hydroxide (KOH) and may also contain a second surfactant—whether anionic (e.g., ADS, SDS), cationic (e.g., CTAB, CTAC), or neutral (e.g., Triton X-100, Tween 20). This step culminates in the generation of a bright chemiluminescence signal, indicative of the presence and quantity of the target immunocomplexes.

This approach incorporates optimized surfactant combinations within the wash buffer to promote effective micelle formation, enhancing immunocomplex dispersion. Additionally, the use of the graphene-based material (e.g., GO), H₂O₂, and specific acid(s) in the first reagent finely tunes the reaction conditions. The subsequent addition of the second reagent, rich in NaOH or KOH along with selected surfactants, triggers a robust chemiluminescence response. This meticulously designed sequence not only improves signal intensity but also ensures the assay's sensitivity and precision over a wider range of analyte concentrations, offering substantial advancements in the field of immunoassay diagnostics.

The optimal conditions for variables, like the choice of surfactant in PBS (or TBS) for the wash buffer, concentrations of components in the first reagent, and the base concentration (e.g., NaOH, KOH) in the second reagent, depend on the type of surfactant used in the second reagent.

### First Embodiment

### The Optimal Conditions for Variables When Using CTAC (or CTAB) in the Second Reagent.

The concentration range for the first surfactant (e.g., Triton X-100, Tween 20, SDS) in PBS (or TBS) in the wash buffer can be 0.05 to 0.3%, more preferably, 0.05 to 0.1%. Within this range, the first surfactant alone or in combination with PBS (or TBS) significantly enhances the Relative Chemiluminescence Intensity (RCL) by stabilizing acridinium ester-labeled immunocomplexes and high-energy intermediates. This stabilization can lead to a nearly threefold increase in RCL when compared with a case where PBS or TBS are used alone without the first surfactant.

The wash buffer, formulated with PBS and/or TBS and including the first surfactant—whether anionic (e.g., ADS, SDS), cationic (CTAB, CTAC), or neutral (Triton X-100, Tween 20)—can be used multiple times to directly wash immunocomplexes formed from immunoreaction. Additionally, up to 50 μl of this wash buffer can be retained in the reaction vessel containing the immunocomplexes after the washing process to boost the effectiveness of acridinium ester chemiluminescence without introducing background noise. The use of the first surfactant in the wash buffer not only ensures the thorough removal of unbound components but also contributes to the stabilization of the immunocomplexes, facilitating an optimal condition for the chemiluminescence reaction.

Acridinium ester-labeled immunocomplexes can exhibit remarkable stability for up to an hour at room temperature in the wash buffer including the first surfactant, such as TBST and TBSS. This contrasts with their rapid degradation in TBS alone. The stability likely stems from anionic (e.g., ADS, SDS), cationic (CTAB, CTAC), or neutral (Triton X-100, Tween 20) surfactant effectively dispersing and forming micelles around the immunocomplexes. This can allow for temporary storage of immunocomplexes in these specific wash buffers before quantification, offering flexibility in assay workflow.

The graphene-based material (e.g., GO) can enhance the normalized CL intensity when used at a concentration of 0.25 to 4 μg/ml, 0.5 to 4 μg/ml, 0.6 to 3 μg/ml, 0.8 to 2 μg/ml, 0.9 to 1.5 μg/ml or 1 μg/ml in the first reagent. The graphene-based material can facilitate the dispersion and stabilization of acridinium ester-labeled immunocomplexes when used together with the wash buffer (e.g., TBS with SDS (TBSS)), and can significantly narrow the error range.

The hydrogen peroxide (H₂O₂) can be included in the first reagent at a concentration range of 2.5 to 40 mM, 3 to 30 mM, 5 to 20 mM, 7 to 15 mM or 10 mM to achieve optimal performance for the chemiluminescence reaction when used together with the second reagent (e.g., CTAC and NaOH), ensuring undetectable background noise and maximizing signal clarity. This range can accommodate various experimental needs, facilitate adjustments to enhance signal strength or accommodate different assay sensitivities.

The acid (e.g., citric acid) can significantly boost the normalized CL intensity at a concentration of 1 to 50 mM. This can suggest that optimal chemiluminescence is achieved when the acid (e.g., citric acid) in the first reagent is effectively neutralized by KOH in the second reagent, creating the most favorable conditions for the reaction. Despite this optimization for the acid, the assay can be versatile, allowing for the analysis of immunocomplexes within a second surfactant (e.g., CTAC or CTAB) concentration range of 3 to 30 mM in the second reagent. This broad range provides the flexibility to fine-tune the assay conditions to meet specific experimental requirements or to optimize the assay's performance for different analyte concentrations. Various acids, including acetic acid, ascorbic acid, formic acid, nitric acid, and oxalic acid, can be also used as alternatives to citric acid.

The second surfactant, e.g., CTAC (or CTAB) in the second reagent can play a pivotal role in boosting the RCL at its optimal concentration of 10 to 50 mM, 20 to 45 mM, 25 to 35 mM, or 30 mM, underlining its importance in stabilizing high-energy intermediates necessary for producing bright chemiluminescence. This concentration can be identified as particularly effective for enhancing assay sensitivity and clarity. This range can accommodate varying experimental needs, enabling researchers to adjust the assay conditions for optimal performance across different studies or to adapt to the specific requirements of diverse analyte concentrations.

The base (e.g., NaOH or KOH) can be utilized at a concentration of 25 to 150 mM, 30 to 120 mM, 40 to 80 mM, or 50 mM, offering a pronounced enhancement in chemiluminescence for the quantification of acridinium-ester labeled immunocomplexes in TBSS. This concentration can ensure a balanced environment for the chemiluminescence reaction, leading to increased sensitivity and specificity of the assay. The assay's flexibility is further demonstrated by its broad NaOH concentration range, from 25 to 150 mM, allowing for adjustments to suit various experimental conditions and to optimize signal intensity.

The calibration curve for the wash buffer (e.g., TBSS) can exhibit a steeper slope relative to the use of TBS or PBS alone, attributed to the enhanced dispersion provided by the first surfactant, such as SDS. This effect contributes to a significantly expanded dynamic range in the immunoassay system when utilizing TBSS, extending from 1.65 pg/ml to 40 ng/ml. This range is considerably wider than that of TBS alone, which spans from 12.7 pg/ml to 20 ng/ml, enabling the direct quantification of high concentrations of target antigens. Such capability eliminates the need for sample dilution, thereby underscoring the system's increased efficiency and practicality.

By meticulously optimizing key components—the wash buffer, the first surfactant, the graphene-based material (e.g., GO), H₂O₂, the acid (e.g., citric acid), the second surfactant (e.g., CTAC or CTAB), and the base (e.g., NaOH)—significant enhancements in sensitivity, specificity, and the dynamic range of chemiluminescence immunoassays targeting acridinium ester-labeled immunocomplexes can be achieved. This optimization not only enhances the assay's performance but also ensures more reliable and accurate detection and quantification of target analytes, demonstrating the critical importance of fine-tuning assay conditions for optimal outcomes.

### Second Embodiment

### The Optimal Conditions for Using Triton X-100 (Or Tween 20) in the Second Reagent.

According to another embodiment of the present disclosure, a reagent system for acridinium ester chemiluminescence reaction including the wash buffer, the first reagent and the second reagent is provided.

The optimal conditions for variables, like the choice of the first surfactant in PBS and/or TBS for the wash buffer, concentrations of components in the first reagent, and the base concentration (e.g., NaOH, KOH) in the second reagent, may depend on the type of the second surfactant used in the second reagent.

The concentration range for the first surfactant or their mixtures (e.g., Triton X-100,Tween 20, SDS) in PBS (or TBS) for use as a wash buffer can be 0.05 to 0.3%. Within this range, an optimal concentration of 0.05 to 0.1% for the first surfactant in PBS (or TBS) can significantly enhance RCL when combined with the second reagent that includes Triton X-100 and the base (e.g., 200 mM NaOH). The optimal concentration range for the second surfactant, including Triton X-100, can be consistent with the concentration for CTAC. This amplification results from the stabilization of acridinium ester-labeled immunocomplexes and high-energy intermediates, can lead to a nearly threefold boost in RCL. Such stabilization can be essential for augmenting the assay's sensitivity and specificity, thereby ensuring accurate detection of antigens in buffers such as PBS or TBS.

The optimal concentration of the graphene-based material (e.g., graphene oxide (GO)) with the wash buffer (e.g., TBST) can be 1 μg/ml, which is lower than the 2 μg/ml needed for TBS alone. This difference can be likely due to the first surfactant in the wash buffer (e.g., TBST) aiding the graphene-based material in dispersing and stabilizing the acridinium ester-labeled immunocomplexes. In addition, a range of the graphene-based material (GO) concentrations (0.25 to 4 μg/ml) in TBST can still enhance CL intensity with good precision, allowing for flexibility in optimizing the assay's sensitivity and specificity.

The optimal concentration of the acid (e.g., citric acid) for triggering acridinium ester chemiluminescence with a second reagent containing the base (e.g., 200 mM NaOH) and the second surfactant (e.g., Triton X-100) can be 12 mM. Exceeding this concentration may lead to diminished CL intensity due to an imbalance with the base (e.g., NaOH), although adjustments in the base concentration can accommodate higher levels of the acid (e.g., citric acid), with the acid (e.g., citric acid) concentration range of 6 to 24 mM being suitable for sensitive reactions. Notably, the effectiveness of the acid in the reaction is consistent across both Triton X-100 and CTAC, indicating that various types of acids can be used interchangeably when neutralized by the base (e.g., 200 mM NaOH). This flexibility in selecting the acids can allow for the optimization of the chemiluminescence reaction across different surfactant conditions, contributing to the assay's adaptability for precise immunocomplex analysis in the wash buffer containing Tween 20, Triton X-100, SDS, and CTAC.

The optimal H₂O₂ concentration for chemiluminescence with Triton X-100 (or Tween 20) can be 100 mM. This can be significantly higher (10×) than the optimal concentration needed with CTAC (or CTAB), indicating a different reaction mechanism with neutral surfactants. A viable working range for H₂O₂ in this system can be 50 to 300 mM. The higher H₂O₂ requirement with neutral surfactants suggests that immunocomplexes within their micelles need more H2O2 to produce optimal chemiluminescence. This can highlight how surfactant choice influences the chemiluminescence reaction and the need for optimization to achieve the best sensitivity.

The optimal concentration of the second surfactant (e.g., Triton X-100) for acridinium ester chemiluminescence reactions can vary based on the wash buffer composition, with 1% (V/V) being suitable for TBST and 3% required for TBSS and TBSC. The combination of 1% Triton X-100 with 0.1% Tween 20, both neutral surfactants, in TBST can synergistically enhance chemiluminescence. In contrast, TBSS and TBSC, which include anionic (SDS) and cationic (CTAC) surfactants, can necessitate a higher Triton X-100 concentration of 3% to achieve optimal interaction and assay performance. The concentration range of Triton X-100 in the second reagent for enhancing immunoassay efficiency can span from 1% to 3% for wash buffers containing diverse surfactant types, including anionic (e.g., ADS, SDS), cationic (e.g., CTAB, CTAC), and neutral (e.g., Triton X-100, Tween 20). Furthermore, it can be feasible to use 0.5% Triton X-100 in the second reagent with 0.1% TBST for immunoassays, albeit with a slightly lower normalized CL intensity compared to 1%. Overall, Triton X-100 concentrations ranging from 0.5% to 5% can be viable for acridinium ester chemiluminescence immunoassays across wash buffers with anionic, cationic, or neutral surfactants, offering considerable flexibility in assay design to optimize sensitivity and specificity.

The optimal concentration of the base (e.g., NaOH) for acridinium ester chemiluminescence immunoassays using Triton X-100 in the second reagent can be 200-400mM. This can be significantly higher (4-8 times) than the optimal concentration needed with CTAC (or CTAB). This difference can indicate that Triton X-100 necessitates a distinct reaction mechanism compared to CTAC or CTAB. For immunoassays using second reagents with neutral surfactants like Triton X-100 or Tween 20, a broader NaOH (or KOH) concentration range of 100-400 mM can provide flexibility for optimizing assay performance and achieving the brightest chemiluminescence signal.

The use of Triton X-100 in NaOH as the second reagent can produce results very similar to those with CTAC in KOH. Notably, TBSS can offer advantages over TBS. It can have enhanced sensitivity with a lower limit of detection (LOD) of 1.25 pg/ml in TBSS compared to 11.9 pg/ml in TBS. TBSS also has a wider dynamic range, handling a much broader range of analyte concentrations (1.25 pg/ml to 40 ng/ml) without the need for dilution. Importantly, the TBSS system can work well with various surfactants (both cationic and neutral) when paired with an alkaline solution. Overall, TBSS offers flexibility, increased sensitivity, and a wider dynamic range, making it a valuable tool for immunoassay development.

Preservatives are essential for extending the shelf life of immunoassay wash buffers. Sodium azide, 5-bromo-5-nitro-1,3-dioxane, and a mixture of 5-chloro-2-methyl-4-isothiazolin-3-one with 2-methyl-4-isothiazolin-3-one can be effective preservatives without significantly quenching chemiluminescence signals when used at low concentrations (around 0.02%). However, higher concentrations of these preservatives, especially sodium azide (above 0.02%) and the isothiazolinone mixture (at 4%), can start to quench the signal. Therefore, optimizing preservative concentration is crucial to ensure wash buffer stability while maintaining strong chemiluminescence for accurate immunoassays.

According to an example of the present disclosure, a sample volume of 25 μl wash buffer (e.g., phosphate-buffered saline with tween 20 (PBST), phosphate-buffered saline with SDS (PBSS), phosphate-buffered saline with CTAC (PBSC), tris-buffered saline with tween 20 (TBST), tris-buffered saline with SDS (TBSS), tris-buffered saline with CTAC (TBSC)) with acridinium ester-labeled immunocomplexes, can be combined with 100 μl of the first reagent containing the graphene-based material (e.g., GO), H₂O₂, and the acid. This mixture can be incubated for 5-60 seconds in a borosilicate test tube to ensure uniform dispersion of the acridinium ester-labeled immunocomplexes. Following this incubation, 300 μl of the second reagent containing the second surfactant (e.g., cationic (e.g., CTAC) or neutral (e.g., Triton X-100) surfactant) and the base (e.g., NaOH or KOH) can be added to the test tube for immediate chemiluminescence measurement, which can be conducted over 5 seconds.

Hereinafter, preferred examples of the present disclosure, comparative examples compared thereto, and test examples for evaluating the examples are described. However, it will be apparent to those skilled in the art that these examples are merely illustrative of the present disclosure, and various changes and modifications can be made within the scope and technical spirit of the present disclosure, and it goes without saying that such variations and modifications fall within the scope of the appended claims.

### EXAMPLES

Acridinium ester chemiluminescence immunoassays involve the sequential addition of first and second reagents to reaction vessels containing acridinium ester-labeled immunocomplexes in one of various wash buffers, such as PBST, PBSS, PBSC, TBST, TBSS, TBSC, illustrated in FIG. 1. The intensity of chemiluminescence is influenced by the type of surfactant used—whether anionic (e.g., ADS, SDS), cationic (e.g., CTAB, CTAC), or neutral (e.g., Triton X-100, Tween 20)—as well as by the concentration and properties of the components within the first and second reagents. The incubation period after introducing the first reagent to the reaction vessel is crucial for the proper dispersion and stabilization of the acridinium ester-labeled immunocomplexes, affecting the relative chemiluminescence intensity.

GO aqueous dispersion at 5 mg/ml, obtained from Goographene in Merrifield, VA, was tested at various concentrations to find the optimal conditions that enhance acridinium ester chemiluminescence and manage background noise in the absence of labeled immunocomplexes. Monoclonal CEA antibodies (anti-CEA) were sourced from Fitzgerald Industries International in Acton, MA. These antibodies were conjugated to acridinium ester, provided by Cayman Chemical in Ann Arbor, MI, through a 30-minute reaction conducted at room temperature to produce a conjugate stored in a buffer from Dojindo, Rockville, MD, and refrigerated as a stock solution. This stock was then diluted with various buffers for control sample preparation.

For assay execution, 25 μl of a control sample in a specific surfactant-containing PBS (or TBS) is placed into a borosilicate tube within the detection cell of a Lumat 9507luminometer, equipped with dual syringe pumps for reagent delivery. This equipment, provided by Berthold Inc., administers 100 μl of the first reagent followed by a 5-60 second incubation period. Then, 300 μl of the second reagent is injected, and the chemiluminescence is recorded for 5 seconds, demonstrating the assay's sensitivity to various experimental conditions.

### Example 1

### Variables in acridinium ester chemiluminescence Reaction Operated with Triton X-100 in the Second Reagent

Effect of Neutral Surfactants in Wash Buffer Containing acridinium ester—Labeled Immunocomplexes for acridinium ester chemiluminescence Immunoassays.

TABLE 1
Effect of neutral surfactants added in a control for the acridinium ester chemiluminescence.

| | Neutral surfactant in a control¹ | | Background² | RCL³ |
|---|---|---|---|---|
| 1^{st} reagent: [GO] = 1 μg/ml, [H₂O₂] = 15 mM, | Triton X-100 | 0 | 0 | 16,557 |
| [Citric acid] = 12 mM | | 0.05% | 0 | 43,560 |
| 2^{nd} reagent: [KOH] = 100 mM, [CTAC] = 30 mM | Tween 20 | 0 | 0 | 15,756 |
| | | 0.05% | 0 | 44,096 |

TABLE 1 illustrates that incorporating a neutral surfactant, such as Triton X-100 or Tween 20, into a positive control containing acridinium ester-labeled immunocomplexes in phosphate-buffered saline (PBS) significantly boosts RCL. This enhancement is attributed to the ability of a 0.05% concentration of Triton X-100 or Tween 20, commonly used in immunoassays as a washing solution, to stabilize both the acridinium ester-labeled immunocomplexes and the high-energy intermediates generated upon adding the first and second reagents. Consequently, RCL in controls containing Triton X-100 or Tween 20 is nearly three times higher than in those without these neutral surfactants. Furthermore, no background signal was detected in negative controls, which lack acridinium ester-labeled immunocomplexes, when neutral surfactants were present.

The findings underscore the role of neutral surfactants not only as stabilizers but also as significant enhancers of the chemiluminescence reaction triggered by acridinium ester. This enhancement effect is consistent even when a neutral (e.g., Triton X-100, Tween 20) or anionic (e.g., ADS, SDS) surfactant is added to the second reagent instead of CTAC, suggesting a synergistic effect of the surfactants present in both the control and the second reagent on RCL enhancement.

Volume Effect of PBST for acridinium ester chemiluminescence Immunoassays

TABLE 2
Volume effect of control containing 0.05% Tween
20 in acridinium ester chemiluminescence reaction

| | Volume | | |
|---|---|---|---|
| | (μl) | Background¹ | RCL² |
| 1^{st} reagent: [GO] = 1 μg/ml, | 20 | 0 | 219,323 |
| [H₂O₂] = 15 mM, | 40 | 0 | 405,865 |
| [Citric acid] = 12 mM | 50 | 61 | 497,987 |
| 2^{nd} reagent: [KOH] = 100 mM, | 100 | 99 | 755,588 |
| [CTAC] = 30 mM | | | |

^{1, 2}Background levels and Relative Chemiluminescence Intensity (RCL) for each experimental condition were measured five times to ensure consistency and reliability of the data (Standard deviation: <5.0%).

TABLE 2 demonstrates that RCL increases with the volume of control solution added to a test tube for the analysis of acridinium ester-labeled immunocomplexes. The control solution's volume notably influences the test's sensitivity and specificity, with significant signal enhancement observed as the volume increases. However, when 50 and 100 μL of the control were added, the background signal remained very low. This low background is attributed to the synergistic effect of the combined solutions—control, first, and second—creating an effective mixture that enhances signal detection while minimizing background noise. These findings suggest that a background-free analysis system can be achieved through the strategic addition of a neutral surfactant, such as Tween 20 or Triton X-100, with careful optimization of the control volume. This approach allows for a refined balance between signal amplification and background suppression, leading to an enhanced analytical performance of the acridinium ester chemiluminescence reaction. Such optimization is crucial for developing sensitive and specific assays in clinical diagnostics and research applications, where precise measurement of acridinium ester-labeled immunocomplexes is essential.

Effect of Anionic, Cationic, And Neutral Surfactants in TBS for acridinium ester chemiluminescence Immunoassays

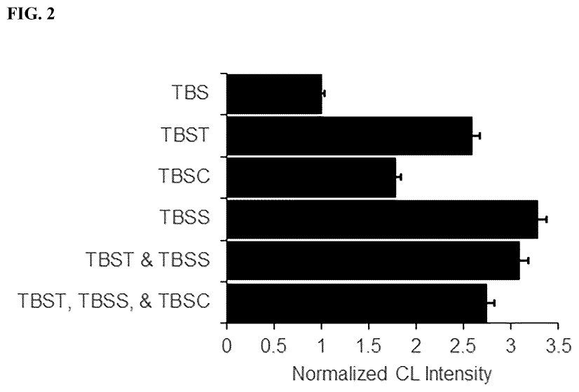

FIG. 2 reveals that the normalized CL intensity in TBS with the inclusion of surfactants—either singly or in combination, such as Tween 20, CTAC, and SDS—exceeds that observed in TBS alone. This enhancement is attributed to the surfactants' ability to rapidly disperse acridinium ester-labeled immunocomplexes, thereby improving their stability. Among the conditions tested, the highest normalized CL intensity was observed in TBSS, facilitated by the rapid interaction between the positively charged acridinium ester and the negatively charged SDS. The findings suggest that surfactants of various types, including anionic (e.g., SDS, ADS), neutral (e.g., Triton X derivatives, Tween 20), and cationic (e.g., CTAC, CTAB), when incorporated into buffers used for immunoassays (e.g., PBS, TBS), can significantly enhance chemiluminescence intensity. This enhancement is likely due to the micelle effect these surfactants induce in the presence of acridinium ester-labeled immunocomplexes, highlighting the critical role of surfactant selection in optimizing the performance of chemiluminescence immunoassays.

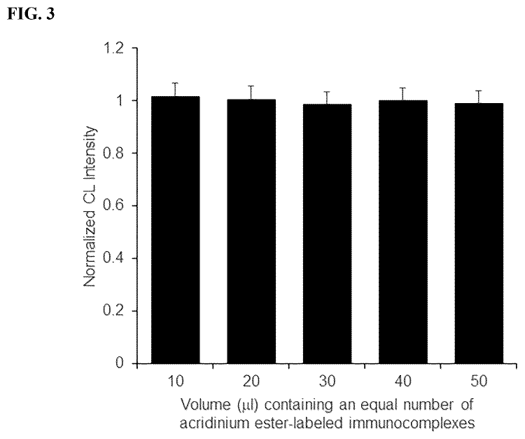

Maintaining a minimal volume of wash buffer in the reaction vessel after its removal—alongside waste or surplus detection antibodies tagged with acridinium ester through the washing process—is achievable. The chemiluminescence intensity generated upon successive additions of the first (100 μl) and second (300 μl) reagents might be influenced by the residual volume of wash buffer within the reaction vessel. Remarkably, in this setup as shown in FIG. 3, the chemiluminescence intensity remained consistent upon adding up to 50 μl of wash buffer, attributed to the constant quantity of acridinium ester-labeled immunocomplexes present in the reaction vessel. This observation underscores that introducing a specific volume of wash buffer, such as PBST, TBST, PBSS, or TBSS, into the reaction vessel to preserve a uniform reaction volume is crucial for the accurate and precise quantification of target antigens in immunoassays. This finding highlights the robustness of the assay against minor variations in wash buffer volume, ensuring reliable antigen detection even with slight procedural variations.

Determination of Incubation Time of acridinium ester—Labeled Immunocomplexes in TBSS and the First Reagent before Adding the Second Reagent to Generate Chemiluminescence

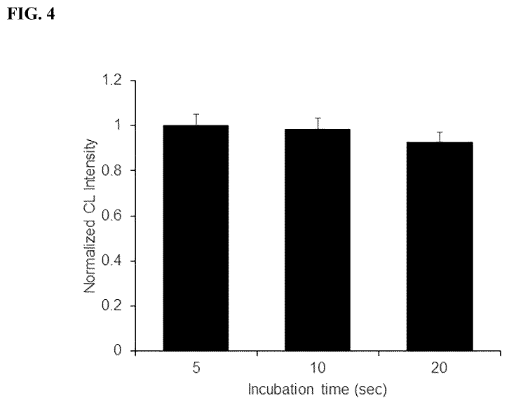

Typically, the incubation period for acridinium ester-labeled immunocomplexes with the first reagent, which does not contain GO, ranges from 1 to 3 minutes. This duration ensures the uniform dispersion of immunocomplexes within the reaction vessel, thereby enhancing chemiluminescence efficiency. However, when the first reagent includes GO, the incubation time can be reduced to at least 20 seconds, due to the GO's hydrophobic nature aiding in the interaction with hydrophobic immunocomplexes in aqueous solutions. Interestingly, as demonstrated in FIG. 4, the optimal incubation time is merely 5 seconds for achieving maximum chemiluminescence brightness. Chemiluminescence intensity decreases for incubation times longer than 5 seconds. The substantial reduction in incubation time can be attributed to the synergistic effect of SDS in TBS and GO, specifically the rapid interaction between the negative charge of SDS and the positive charge of acridinium ester-labeled immunocomplexes, further stabilized by GO.

In conclusion, the necessary incubation time is significantly influenced by the synergistic interactions between the wash buffer (including PBST, PBSS, TBST, and TBSS) containing acridinium ester-labeled immunocomplexes and GO in the first reagent. Under these conditions, the incubation time can range from 5 to 60 seconds, highlighting the importance of optimizing both the composition of the wash buffer and the incubation parameters to enhance the performance of chemiluminescence immunoassays.

Stability of acridinium ester—Labeled Immunocomplexes in Wash Buffer Containing Anionic, Cationic, or Neutral Surfactant

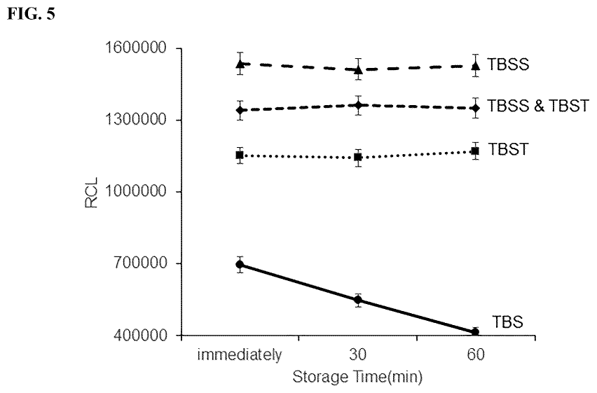

FIG. 5 demonstrates that acridinium ester-labeled immunocomplexes, generated from the immunoreaction between target antigens and antibodies tagged with acridinium ester, exhibit remarkable stability for up to an hour at room temperature in wash buffers like TBST, TBSS, and their combination. This stability contrasts with their rapid degradation observed in TBS. The presence of Tween 20 and SDS in these wash buffers facilitates the broad dispersion of immunocomplexes under micelle conditions, thereby enhancing their stability at room temperature. The findings from FIG. 5 suggest that acridinium ester-labeled immunocomplexes resulting from immunoassay procedures can be temporarily stored for a specific duration (e.g., 1hour) in selected wash buffers without proceeding immediately to sample quantification through the addition of the first and second reagents. However, for maintaining the accuracy in quantifying samples, it's recommended that immunocomplexes in TBS (or PBS) are analyzed promptly after formation. This highlights the importance of choosing appropriate wash buffer compositions to ensure the stability of acridinium ester-labeled immunocomplexes, thereby offering flexibility in the timing of subsequent analysis steps without compromising the assay's accuracy.

Determination of GO Concentration Range for acridinium ester chemiluminescence Immunoassays using the Second Reagent Containing CTAC

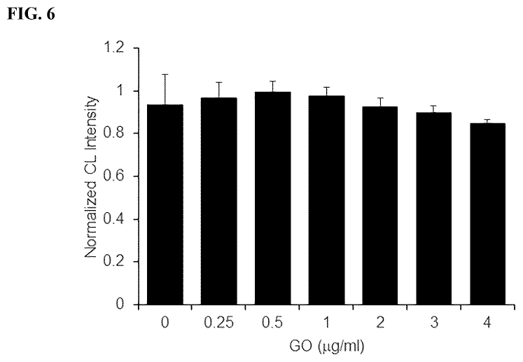

FIG. 6 reveals the role of GO in facilitating the dispersion and stability of acridinium ester-labeled immunocomplexes in TBSS, leveraging the hydrophobic properties of GO. When the first reagent was introduced without GO, although it could induce bright chemiluminescence, the variability in the measurements was relatively high, indicated by a wide error margin (±12%). However, upon incorporating GO at concentrations up to 0.5 or 1 μg/ml into the first reagent, there was a noticeable enhancement in the normalized chemiluminescence (CL) intensity, coupled with a significant narrowing of the error range (±4%). This improvement is attributed to the synergistic effects arising from the interaction between TBSS and GO, which aid in more effectively dispersing and stabilizing the immunocomplexes. Conversely, adding GO beyond 1 μg/ml led to a reduction in normalized CL intensity, a consequence of the quenching effect caused by the excess GO, despite further addressing variability concerns.

The observations from FIG. 6 indicate the optimal concentration of GO at 1 μg/ml within the first reagent to maximize chemiluminescence intensity while ensuring high levels of accuracy and precision in sample analysis. This highlights the critical balance between enhancing assay performance and avoiding potential negative effects of reagent overconcentration. Thus, careful optimization of GO concentration is essential for improving the sensitivity and reliability of immunoassays, demonstrating the importance of fine-tuning reagent formulations to achieve the best possible assay outcomes.

Determination of H₂O₂ Concentration Range for acridinium ester chemiluminescence Immunoassays Using the Second Reagent Containing CTAC

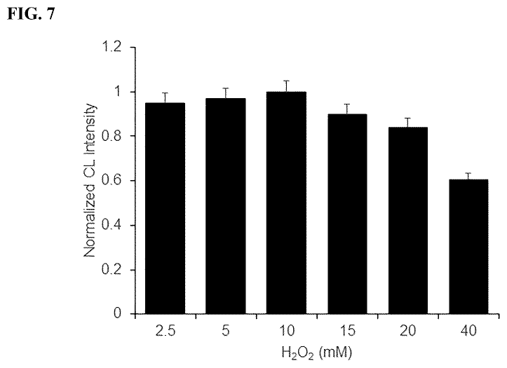

FIG. 7 illustrates that for acridinium ester chemiluminescence reactions involving acridinium ester-labeled immunocomplexes in TBSS, the addition of GO and citric acid in the first reagent, and a second reagent comprising CTAC and NaOH, the optimal H₂O₂ concentration was identified as 10 mM. Furthermore, under these optimal conditions, background noise was undetectable even when using a highly sensitive luminometer (Lumat 9507, Berthold Inc.), in the absence of acridinium ester-labeled immunocomplexes. These results indicate that an immunoassay system designed to minimize background interference by utilizing a low concentration of H₂O₂ in the first reagent can achieve exceptional levels of accuracy, precision, sensitivity, and reliability. This optimization underscores the importance of carefully selecting reagent concentrations to enhance the performance of chemiluminescence immunoassays, ensuring that they can provide highly accurate and reliable results even at low analyte concentrations.

Determination of Acidic Compound Concentration Range for acridinium ester chemiluminescence Immunoassays Using the Second Reagent Containing CTAC

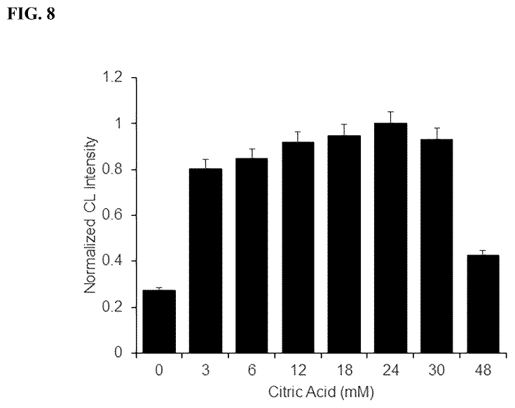

FIG. 8 demonstrates the critical function of acidic components, such as citric acid, within the first reagent for acridinium ester chemiluminescence reactions. The inclusion of citric acid significantly elevates normalized chemiluminescence (CL) intensity, facilitating the even dispersion of acridinium ester-labeled immunocomplexes when mixed with TBSS and the first reagent. An upward trend in normalized CL intensity was observed with increasing citric acid concentration, peaking at 24 mM. Beyond this concentration, the addition of higher levels of citric acid resulted in a decrease in normalized CL intensity when compared to the peak intensity achieved at 24 mM. These results indicate that an optimal acidic environment, achieved with 24 mM citric acid in the first reagent and neutralized by 100 mM KOH in the second reagent, creates the most favorable conditions for generating the brightest chemiluminescence.

Further investigation confirmed that the optimal concentration for other acidic reagents, such as oxalic acid, formic acid, nitric acid, acetic acid, and ascorbic acid, mirrors that of citric acid in enhancing the acridinium ester chemiluminescence reaction. This consistency is attributed to the neutralization effect when 24 mM of these acidic reagents in the first reagent encounters 100 mM NaOH in the second reagent. This insight underscores the importance of precisely calibrating the acidic balance in the first reagent to ensure optimal pH conditions for the chemiluminescence reaction, thereby enhancing the assay's sensitivity, accuracy, and overall performance with various acidic reagents in combination with CTAC in NaOH as the second reagent. Based on the results shown in FIG. 8, The acidic compound for acridinium ester chemiluminescence immunoassay using CTAC in the second reagent has a wide concentration range of 3-30 mM.

Determination of CTAC Concentration for acridinium ester chemiluminescence Immunoassays Using TBS Containing Tween 20 or SDS

FIG. 9 demonstrates that the inclusion of CTAC with KOH in the second reagent notably enhances the RCL, primarily due to its critical role in stabilizing high-energy intermediates within micelle formations. This stabilization effectively shields the reactive intermediates from interference, facilitating the production of bright chemiluminescence. The optimal CTAC concentration identified for maximally boosting RCL in wash buffers like TBSS and TBST was 30 mM. The comparison revealed that RCL in wash buffer is more intense than that in TBS, highlighting the synergistic effect arising from the combination of surfactants such as SDS (or Tween 20) in TBS and CTAC in the second reagent. Moreover, the results suggest that the acridinium ester chemiluminescence reaction's efficiency can be finely tuned across a broad spectrum of CTAC concentrations, ranging from 10 to 50 mM in KOH (or NaOH), allowing for adaptable optimization of the immunoassay conditions to achieve the best possible sensitivity and specificity.

Determination of NaOH Concentration for acridinium ester chemiluminescence Immunoassays with Wash Buffer Containing acridinium ester-labeled Immunocomplexes and CTAC in the Second Reagent

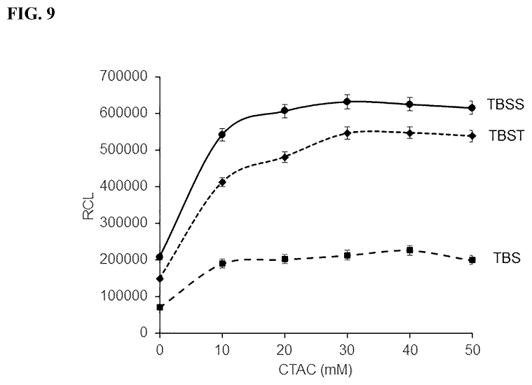

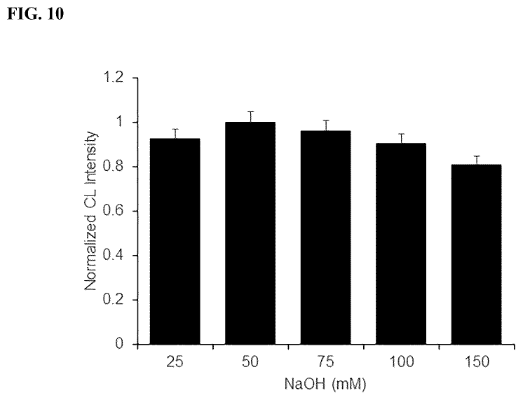

FIG. 10 showcases the dependency of the acridinium ester chemiluminescence reaction's efficiency on the concentration of NaOH, which is instrumental in setting the pH of the final solution resulting from the combination of TBSS, the first, and the second reagents. A concentration of 50 mM NaOH emerged as optimal for producing the highest normalized CL intensity, though a broad range of NaOH concentrations, from 25 to 150 mM, is viable for the quantification of acridinium-ester labeled immunocomplexes in TBSS with pronounced chemiluminescence. The study also verified that background noise remained non-existent across varying concentrations of NaOH, underscoring the reaction's specificity under the given conditions. Importantly, the selection of the ideal NaOH (or KOH) concentration is subject to variation, contingent upon the acidic component's concentration within the first reagent, highlighting the nuanced balance required between reagent concentrations to optimize the chemiluminescence output effectively.

Linear Calibration Curves for TBSS and TBS in acridinium ester chemiluminescence Immunoassays Operated with CTAC in the Second Reagent

TABLE 3
Expected RCL of acridinium ester-labeled
immunocomplexes in TBS and TBSS

| Acridinium ester-labeled | RCL | RCL | |
|---|---|---|---|
| Immunocomplex (pg/ml) | in TBS¹ | in TBSS² | RCL_{TBSS}/RCL_{TBS} |
| 312.5 | 3816 | 12404 | 3.25 |
| 156.3 | 1739 | 6292 | 3.62 |
| 78.1 | 792 | 3192 | 4.03 |
| 39.1 | 361 | 1619 | 4.49 |
| 19.5 | 164 | 821 | 4.99 |
| 9.8 | 75 | 417 | 5.56 |
| 4.9 | 34 | 211 | 6.19 |
| 2.4 | 16 | 107 | 6.89 |
| 1.2 | 7 | 54 | 7.67 |
| 0.6 | 3 | 28 | 8.54 |

¹The RCL for immunocomplexes labeled with acridinium ester at a concentration of 312.5 pg/ml in TBS was measured following the introduction of the first and second reagents. Subsequent RCL values for concentrations of acridinium ester-labeled immunocomplexes below 312.5 pg/ml were calculated using Equation 1.
²The RCL for immunocomplexes labeled with acridinium ester at a concentration of 312.5 pg/ml in TBST was

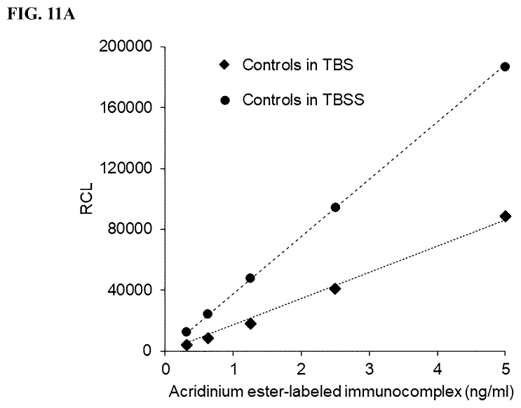

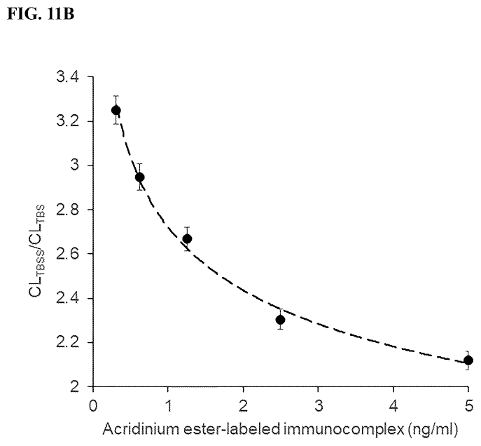

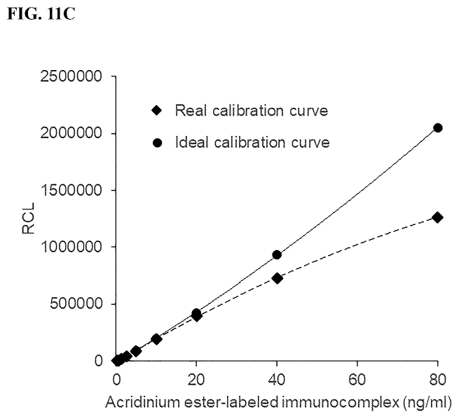

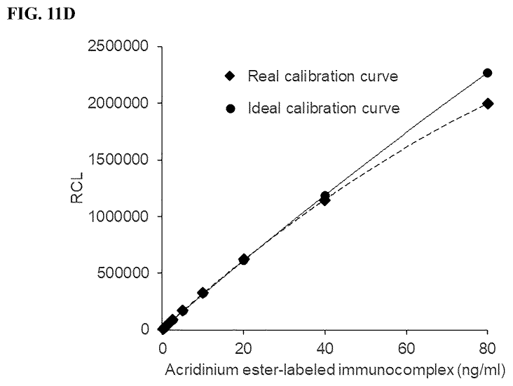

FIG. 11A illustrates that the linear calibration curve (y =39,313x) in TBSS has a larger slope compared to that (y=13,015x) in TBS, attributable to the presence of 0.1% SDS in TBSS which facilitates even and broad dispersion of acridinium ester-labeled immunocomplexes. This dispersal enables a more intense chemiluminescence emission as opposed to the less stable aggregates formed in TBS. The intercepts of both the linear calibration curves for TBS and TBSS were established at zero, reflecting the absence of detectable background signals in measurements devoid of acridinium ester-labeled immunocomplexes.

However, the rate of increase in RCL for TBSS was 97%, compared to 119% in TBS when the concentration of acridinium ester-labeled immunocomplexes was doubled. Consequently, as the concentration of acridinium ester-labeled immunocomplexes increased, the ratio of RCL in TBSS to RCL in TBS, RCLTBSS/RCLTBS, decreased, as depicted in FIG. 11B. Inversely, RCLTBSS/RCLTBS proportionately increased as the concentration of acridinium ester-labeled immunocomplexes decreased, as illustrated in Table 3. These findings suggest that the immunoassay system without background interference, utilizing acridinium ester-labeled immunocomplexes in TBSS, exhibits greater sensitivity than that in TBS. The RCL values for TBS and TBSS, as shown in Table 3, were calculated using equations 1 and 2, respectively.

$${RCL}_{lower} = {RCL}_{higher} / 2.19 \qquad ( 1 )$$

$${RCL}_{lower} = {RCL}_{higher} / 1.97 \qquad ( 2 )$$

RCLlower represents the RCL value at an acridinium ester-labeled immunocomplex concentration that is half of the RCLhigher concentration. For instance, the RCL values for TBS and TBSS at 312.5 pg/ml of acridinium ester-labeled immunocomplexes were measured directly, as depicted in FIG. 11A, and these values serve as the RCLhigher reference points for estimating RCLlower for 156.3 pg/ml using equations 1 and 2. Accordingly, RCLlower in TBS is 2.19 times less than RCLhigher, while in TBSS, it is 1.97 times less. This discrepancy is due to the differing reduction ratios—119% for TBS compared to 97% for TBSS. Consequently, RCLTBSS/RCLTBS for 156.3 pg/ml is greater than that for 312.5 pg/ml. Assuming the concentration that yields an

RCL of 100 is the quantitative Limit of Detection (LOD) in a system without background interference, the LOD for TBS and TBSS would be 12.7 pg/ml and 1.65 pg/ml, respectively. Therefore, the LOD in TBSS is approximately 7.7 times lower than in TBS.

Typically, RCL in the presence of relatively high concentrations of acridinium ester-labeled immunocomplexes may be lower than expected from the linear calibration curve equation derived at lower concentrations. This is due to the self-quenching effect that arises from the aggregation of hydrophobic immunocomplexes in an aqueous solution such as TBS. As indicated in FIG. 11C, the RCL measured at an acridinium ester-labeled immunocomplex concentration of 20 ng/ml is marginally lower than predicted by the linear calibration curve equation presented in FIG. 11A. Moreover, the RCL values measured at 40 and 80 ng/ml of acridinium ester-labeled immunocomplexes show a complete deviation from the expected values.

FIGS. 11C and 11D illustrate that the deviation between the measured and calculated RCL at 80 ng/ml of acridinium ester-labeled immunocomplexes is considerably smaller in TBSS than in TBS. Furthermore, the RCL measured at 40 ng/ml in TBSS coincides with the anticipated value, suggesting that the higher concentration of acridinium ester-labeled immunocomplexes is more uniformly dispersed by SDS in TBSS, mitigating the self-quenching effect. These results suggest that the immunoassay system using TBSS can directly quantify samples with high concentrations of target antigens without the need for labor-intensive procedures such as sample dilution.

In conclusion, the dynamic range of the immunoassay system employing TBSS spans from 1.65 pg/ml to 40 ng/ml, which is significantly broader compared to that in TBS, which extends from 12.7 pg/ml to 20 ng/ml. This extended range in TBSS facilitates the direct quantification of samples with high target antigen concentrations and enhances the efficiency and utility of the immunoassay.

### Example 2

### Variables in acridinium ester chemiluminescence Reaction Operated with Triton X-100 in the Second Reagent

### Determination of Optimal Concentration Of Surfactant in TBS (or PBS) as Wash Buffer

The ideal surfactant concentration range for wash buffers like PBS or TBS, incorporating mixtures such as Triton X-100, Tween 20, and SDS, is established between 0.05 to 0.3%. Specifically, a concentration of 0.05 to 0.1% surfactant significantly increases Relative Chemiluminescence Intensity (RCL) when used alongside a second reagent containing Triton X-100 and 200 mM NaOH. This optimal concentration is consistent across surfactants, including CTAC. The enhancement in RCL is attributed to the effective stabilization of acridinium ester-labeled immunocomplexes and high-energy intermediates, facilitating up to a threefold increase in RCL. This stabilization plays a crucial role in improving the sensitivity and specificity of the assay, crucial for the precise detection of antigens in PBS or TBS buffers.

### Determination of Optimal Concentration of GO in the Presence of Triton X-100

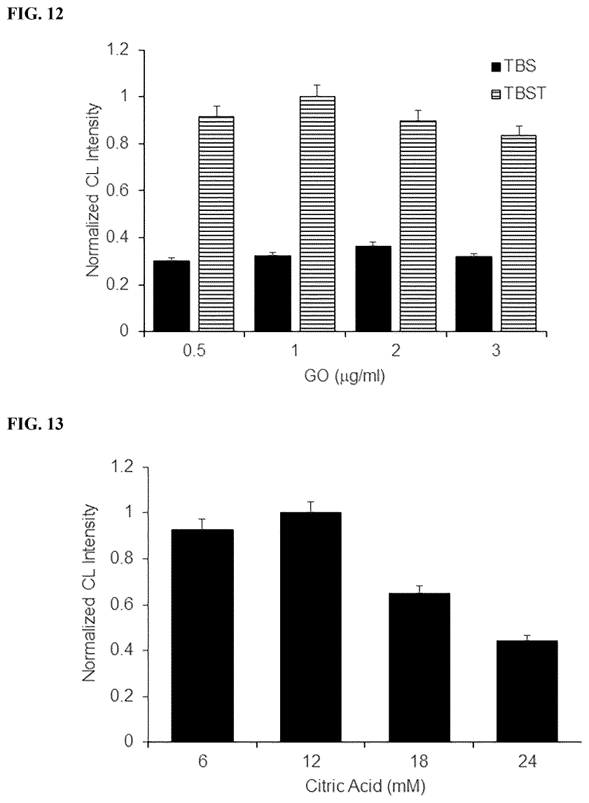

FIG. 12 reveals that the optimal graphene oxide (GO) concentration for TBST is 1 μg/ml, contrasting with the 2 μg/ml required for TBS. This discrepancy arises because GO's ability to uniformly disperse acridinium ester-labeled immunocomplexes and stabilize them is complemented by the surfactant presence in TBS. Consequently, the required GO concentration for safeguarding immunocomplexes in the aqueous environment of TBST is less than in TBS alone. Additionally, FIG. 12 demonstrates that a range of GO concentrations (0.5-3 μg/ml) in TBST can effectively enhance normalized chemiluminescence (CL) intensity while maintaining good precision, offering flexibility in assay optimization to achieve the desired balance between sensitivity and specificity.

### Determination of Optimal Concentration of Acidic Chemical in the Presence of Triton X-100

FIG. 13 demonstrates that the optimal citric acid concentration for acridinium ester chemiluminescence, triggered with a second reagent containing 200 mM NaOH and Triton X-100, is established at 12 mM. Beyond this concentration, a significant decrease in normal CL intensity is observed, attributed to an imbalance between citric acid and the 200 mM NaOH, which is not conducive to producing bright chemiluminescence. Nonetheless, employing a higher citric acid concentration alongside an increased NaOH concentration, exceeding 200 mM, can achieve optimal conditions for chemiluminescence. Therefore, a citric acid concentration range of 6 to 24 mM is viable for conducting highly sensitive acridinium ester chemiluminescence reactions. It's noteworthy that the suitable concentration range of citric acid varies when Triton X-100 (or Tween 20) is used instead of CTAC, as previously shown in FIG. 8, indicating the importance of adjusting reagent concentrations based on the specific components of the chemiluminescence assay to optimize performance.

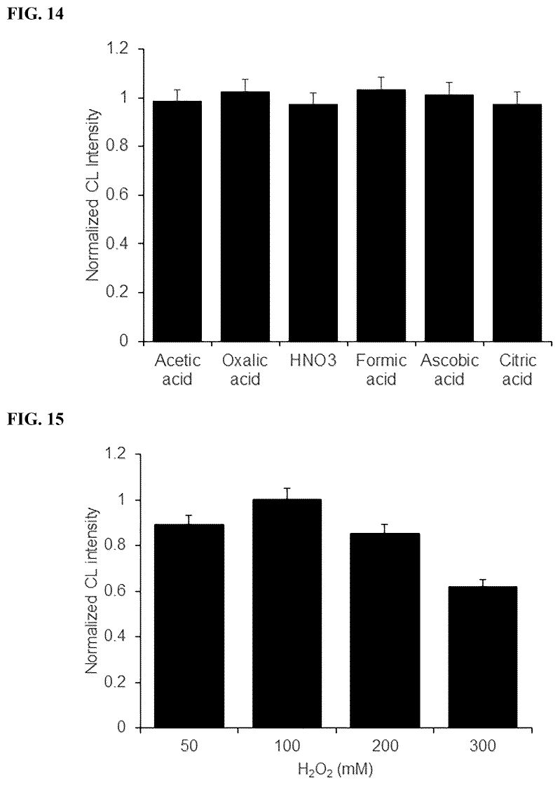

FIG. 14 reveals that the normalized CL intensity in the presence of Triton X-100 does not vary with the type of acidic compound used, as a 12 mM concentration of any acidic compound is effectively neutralized by the addition of 200 mM NaOH in the second reagent. This observation is consistent across experiments using CTAC, underscoring the flexibility in choosing acidic compounds for the chemiluminescence reaction. Consequently, a variety of acidic compounds can be utilized to fine-tune the reaction environment, enhancing the acridinium ester chemiluminescence for analyzing immunocomplexes in wash buffers that include Tween 20, Triton X-100, SDS, and CTAC. This versatility in acidic compound selection allows for the optimization of assay conditions to achieve maximum sensitivity and specificity across different wash buffer formulations.

Concentration Range of H₂O₂ for acridinium ester chemiluminescence Triggered by the Second Reagent Containing Triton X-100

FIG. 15 shows that the brightest chemiluminescence in the presence of Triton X-100 is achieved with an optimal H₂O₂ concentration of 100 mM. This concentration is notably 10 times higher than the optimal concentration required when using CTAC, suggesting a distinct reaction mechanism with neutral surfactants like Triton X-100 and Tween 20. A H₂O₂ concentration range from 50 to 300 mM is considered viable for developing an immunoassay system tailored to acridinium ester chemiluminescence detection. This adaptation indicates that acridinium ester- labeled immunocomplexes, when encapsulated within micelles formed by neutral surfactants, require a higher H₂O₂ concentration, up to 100 mM, to efficiently produce bright chemiluminescence. This observation underscores the significance of surfactant choice and its interaction with H₂O₂ concentration in optimizing the chemiluminescence reaction for precise and sensitive detection of target immunocomplexes.

Concentration range of Triton X-100 for acridinium ester chemiluminescence reaction in the Presence of Wash Buffer Containing acridinium ester-labeled Immunocomplexes

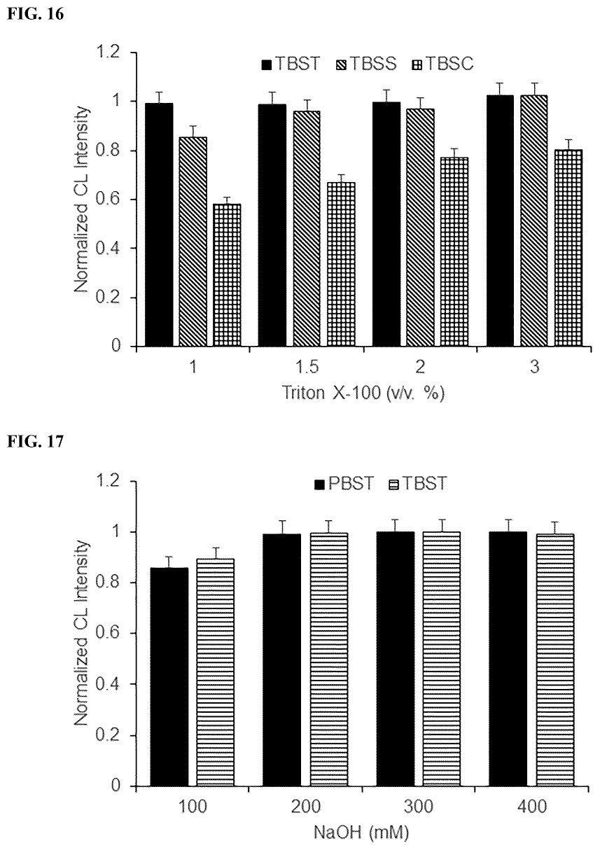

FIG. 16 highlights how the optimal Triton X-100 concentration for acridinium ester chemiluminescence reactions varies with the type of wash buffer used. A 1% Triton X-100 concentration is sufficient for assays in TBST, while a higher concentration of 3% is optimal for reactions in TBSS and TBSC. Given Tween 20′s role as a neutral surfactant similar to Triton X-100, combining 1% Triton X-100 with 0.1% Tween 20 can synergistically enhance chemiluminescence brightness and the sensitivity of the immunoassay. Conversely, for TBSS and TBSC, which involve anionic (SDS) and cationic (CTAC) surfactants, a higher Triton X-100 concentration of up to 3% is required to effectively interact and perform advanced immunoassays. The range of 1-3% Triton X-100 proves versatile, supporting enhanced assay efficiency with wash buffers containing a variety of surfactant types. Additionally, extending Triton X-100 concentration up to 5% offers further flexibility in assay design, especially for buffers containing cationic surfactants like CTAB and CTAC. It's also noted that 0.5% Triton X-100 in the second reagent, combined with 0.1% TBST, is practically effective, with normalized CL intensity slightly lower than at 1%. Thus, a broad concentration range of 0.5-5% Triton X-100 is viable for acridinium ester chemiluminescence immunoassays, accommodating wash buffers with anionic, cationic, or neutral surfactants. This flexibility allows for the tailoring of assay conditions to maximize sensitivity and specificity across a wide array of immunoassay applications.

Determination of NaOH Concentration Range for acridinium ester chemiluminescence Reaction for the Quantification of acridinium ester-labeled Immunocomplexes in Wash Buffer

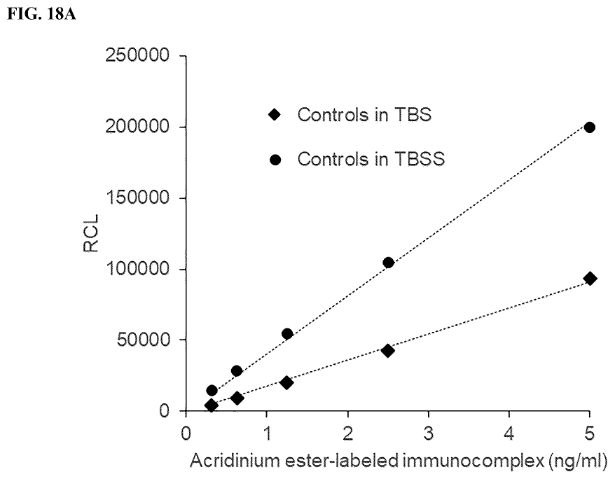

FIG. 17 reveals that for acridinium ester chemiluminescence immunoassays utilizing a second reagent with Triton X-100, the optimal sodium hydroxide (NaOH) concentration range is between 200 to 400 mM. This range is significantly higher—4 to 8 times—than the optimal concentration needed in the presence of cetyltrimethylammonium chloride (CTAC), as detailed in FIG. 10. This discrepancy highlights a distinct mechanism of action for the acridinium ester chemiluminescence reaction when Triton X-100 is involved compared to CTAC. Consequently, based on these insights, the adaptable concentration range for NaOH (or KOH) in such immunoassays extends from 100 to 400 mM, allowing for fine-tuning based on the specific surfactant, such as Triton X-100 and Tween 20, used in the second reagent to optimize assay performance and maximize chemiluminescence output.

Linear Calibration Curves for TBSS and TBS in acridinium ester chemiluminescence Immunoassays Operated with Triton X-100 in the Second Reagent

TABLE 4
Expected RCL of acridinium ester-labeled
immunocomplexes in PBS and PBSS

| Acridinium ester-labeled | RCL | RCL | |
|---|---|---|---|
| Immunocomplex (pg/ml) | in TBS¹ | in TBSS² | RCL_{TBSS}/RCL_{TBS} |
| 312.5 | 4342 | 14794 | 3.41 |
| 156.3 | 1978 | 7504 | 3.79 |
| 78.1 | 901 | 3807 | 4.22 |
| 39.1 | 411 | 1931 | 4.70 |
| 19.5 | 187 | 979 | 5.23 |
| 9.8 | 85 | 497 | 5.83 |
| 4.9 | 39 | 252 | 6.49 |
| 2.4 | 18 | 128 | 7.22 |
| 1.2 | 8 | 65 | 8.04 |
| 0.6 | 4 | 33 | 8.95 |

¹The RCL for immunocomplexes labeled with acridinium ester at a concentration of 312.5 pg/ml in TBS was measured following the introduction of the first and second reagents. Subsequent RCL values for concentrations of acridinium ester-labeled immunocomplexes below 312.5 pg/ml were calculated using Equation 3.
²The RCL for immunocomplexes labeled with acridinium ester at a concentration of 312.5 pg/ml in TBST was measured following the introduction of the first and second reagents. Subsequent RCL values for concentrations of acridinium ester-labeled immunocomplexes below 312.5 pg/ml were calculated using Equation 4.

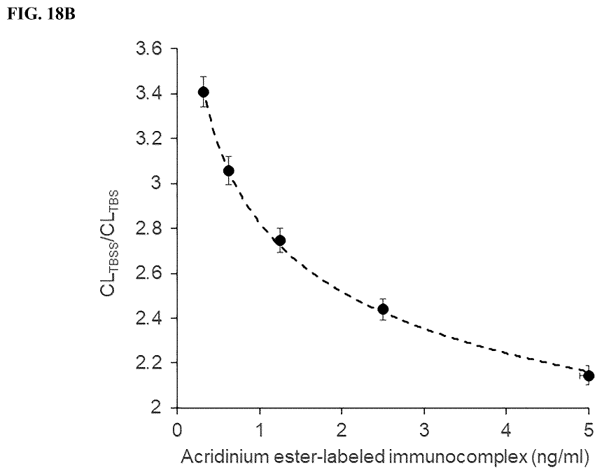

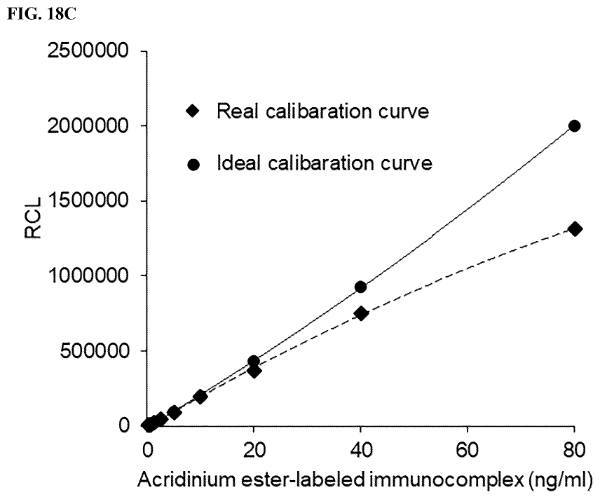

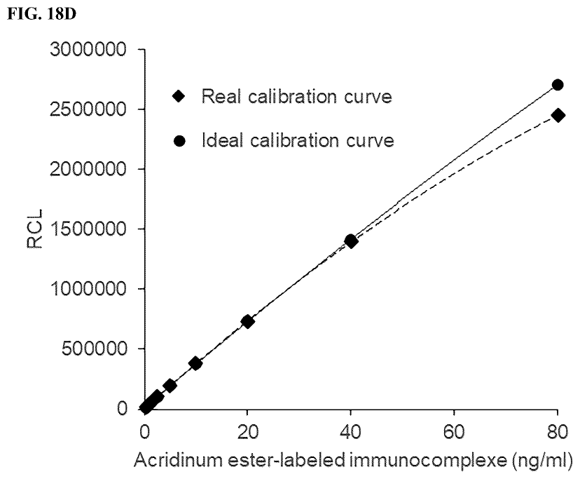

The findings presented in FIGS. 18A to 18D and Table 4, utilizing Triton X-100 in NaOH as the second reagent, align closely with those observed in FIGS. 11A to 11D and TABLE 3,where CTAC in KOH was used as an alternative second reagent. Specifically, FIG. 18A reveals that the calibration curve's slope in TBSS exceeds that in TBS. Furthermore, FIG. 18B and TABLE 4 demonstrate that the ratio of RCLTBSS/RCLTBS varies with the concentration of acridinium ester-labeled immunocomplexes, indicating that the modulation of RCL—either an enhancement or a reduction—is significantly distinct between TBSS and TBS, at rates of 92% and 115%, respectively. This suggests that the choice of surfactant and alkaline conditions in the second reagent can markedly influence the dispersion and stabilization of acridinium ester-labeled immunocomplexes, potentially affecting the assay's sensitivity and dynamic range. The estimated RCL values for TBS and TBSS listed in Table 4 were derived using equations 3 and 4,respectively, underscoring the importance of selecting appropriate reagent formulations to optimize assay performance, especially in terms of achieving a broader dynamic range and higher sensitivity in detecting target antigens.

$${RCL}_{lower} = {RCL}_{higher} / 2.15 \qquad ( 3 )$$

$${RCL}_{lower} = {RCL}_{higher} / 1.92 \qquad ( 4 )$$

FIGS. 18C and 18D demonstrate a significantly broader dynamic range in TBSS (1.25pg/ml to 40 ng/ml) compared to TBS (11.9 pg/ml to 20 ng/ml), highlighting the enhanced sensitivity and versatility of the TBSS-based immunoassay system. The lower LOD in TBSS, at 1.25 pg/ml, is approximately 10 times less than in TBS, which is 11.9 pg/ml. Additionally, the ability to quantify up to 40 ng/ml in TBSS without necessitating sample dilution underscores its capability to handle a wider spectrum of analyte concentrations effectively.

To conclude, the TBSS system facilitates accurate and precise quantification of immunocomplexes at both trace and relatively high concentrations encountered during the immunoassay process. This system demonstrates compatibility with a variety of surfactants—both cationic, such as CTAC and CTAB, and neutral, like Triton X-100 and Tween 20—when paired with an alkaline solution like NaOH (or KOH) as the second reagent. Such flexibility in reagent choice allows for the optimization of assay conditions to achieve desired sensitivity and specificity levels, making the TBSS approach a versatile tool for immunoassay development and application.

Effect of Preservatives in Wash Buffer for acridinium ester chemiluminescence Immunoassays

TABLE 5
Effect of preservatives in wash buffer

| | Preservatives | % | Background¹ | RCL² |
|---|---|---|---|---|
| Acridinium ester-labeled immunocomplexes in | No | — | 0 | 256,005 |
| TBSS with or without a preservative | Sodium azide | 0.02 | 0 | 258,467 |
| 1^{st} reagent: [GO] = 1 μg/ml, [H₂O₂] = 100 mM, | | 0.04 | 0 | 196,823 |
| [Citric acid] = 12 mM | | 0.08 | 0 | 118,985 |
| 2^{nd} reagent: [KOH] = 200 mM, [Triton X-100] = | 5-Bromo-5-nitro-1,3- | 0.02 | 0 | 262,206 |
| 1.5% | dioxane | | | |
| | Mixture of two | 0.02 | 0 | 257,673 |
| | preservatives³ | 0.2 | 0 | 255,575 |
| | | 4 | 0 | 168,094 |

^{1, 2}Background levels and Relative Chemiluminescence Intensity (RCL) for each experimental condition were measured five times to ensure consistency and reliability of the data (Standard deviation: <5.0%).
³The proportion of 5-chloro-2-methyl-4-isothiazolin-3-one to 2-methyl-4-isothiazolin-3-one is set at 3:1.

To enhance the stability of wash buffers in immunoassays, trace quantities of preservatives or their combinations can be incorporated, taking into account their toxicity and their potential as effective quenchers that can diminish the signals in chemiluminescence immunoassays. Typically, the inclusion of as little as 0.02% of a preservative can significantly prolong the wash buffer's shelf-life. Table 5 demonstrates that sodium azide, 5-bromo-5-nitro-1,3-dioxane, and a blend of 5-chloro-2-methyl-4-isothiazolin-3-one with 2-methyl-4-isothiazolin-3-one can serve as effective preservatives in wash buffers for acridinium ester chemiluminescence immunoassays. This is evidenced by the observation that the RCL with these preservatives matches that of a preservative-free wash buffer, implying no significant quenching effect at these concentrations.

However, the data also indicates that an RCL reduction occurs with higher concentrations of sodium azide; specifically, a concentration above 0.02% in TBSS as the wash buffer. This suggests that there is a critical concentration threshold for sodium azide, beyond which it begins to significantly quench the chemiluminescence reaction.

Moreover, the preservative mixture of 5-chloro-2-methyl-4-isothiazolin-3-one and 2-methyl-4-isothiazolin-3-one exhibits a quenching effect at a 4% concentration in the wash buffer. It's notable that the RCL is notably lower at this high concentration, indicating that the quenching properties of this preservative mixture are concentration-dependent.

The findings highlight the importance of optimizing preservative concentrations to balance the antimicrobial efficacy and the minimization of quenching effects, ensuring both the preservation of wash buffer and the integrity of the chemiluminescence signal in immunoassays. This balance is crucial for reliable assay performance over an extended period, which is a key consideration for commercial and laboratory applications where prolonged storage of reagents is necessary.

What is claimed is:

1. A reagent system for eliciting an acridinium ester chemiluminescence, comprising:
a wash buffer including a first surfactant in one or a combination of a phosphate-buffered saline (PBS) and a tris-buffered saline (TBS);
a first reagent including hydrogen peroxide and an acid, and optionally a first alkali metal salt; and
a second reagent including a base and a second surfactant, and optionally a second alkali metal salt,
wherein the first surfactant and the second surfactant are independently one or more of a cationic surfactant, a neutral surfactant and an anionic surfactant,
wherein the acid has an acid dissociation constant (pKa) of −1.0 to 5.0, and
wherein the base comprises one or more of sodium hydroxide (NaOH), potassium hydroxide (KOH), ammonium hydroxide, tetra-n-ethylammonium hydroxide (TEA), tetra-n-propylammonium hydroxide (TPA), and tetra-n-butylammonium hydroxide (TBA).

2. The reagent system of claim 1, wherein the first surfactant is present in the wash buffer at a concentration of 0.05 to 0.3%.

3. The reagent system of claim 1, wherein the first reagent further comprises a graphene-based material selected from the group consisting of graphene, graphene oxide (GO), and reduced graphene oxide (rGO).

4. The reagent system of claim 1, wherein the acid having acid dissociation constant (pKa) of −1.0 to 5.0 comprises one or more selected from the group consisting of acetic acid, citric acid, formic acid, nitric acid (HNO₃), oxalic acid, ascorbic acid, salicylic acid, hydrogen chloride, tartaric acid, lactic acid.

5. The reagent system of claim 1, wherein the cationic surfactant comprises cetyltrimethylammonium chloride (CTAC) or cetyltrimethylammonium bromide (CTAB).

6. The reagent system of claim 1, wherein the neutral surfactant is one or more polyethylene glycol tert-octylphenyl ether derivatives and/or polyoxyethylenesorbitan monopalmitate derivatives selected from the group consisting of Triton X-45, Triton X-100,Triton X-102, Triton X-114, Triton X-165, Triton X-305, Triton X-405, Triton X-705, Tween 20, Tween 40, Tween 60, Tween 80.

7. The reagent system of claim 1, wherein the anionic surfactant comprises ammonium dodecyl sulfate (ADS) or sodium dodecyl sulfate (SDS).

8. The reagent system of claim 3, wherein the graphene-based material is present in the first reagent at a concentration of more than 0 to 10 μg/ml.

9. The reagent system of claim 1, wherein the hydrogen peroxide is present in the first reagent at a concentration of 2.5 to 50 mM, and
wherein the second surfactant comprises cetyltrimethylammonium chloride (CTAC) or cetyltrimethylammonium bromide (CTAB).

10. The reagent system of claim 1, wherein the hydrogen peroxide is present in the first reagent at a concentration of 10 to 400 mM, and
wherein the second surfactant comprises Triton X-100 or Tween 20.

11. The reagent system of claim 1, wherein the acid is present in the first reagent at a concentration of 1 to 50 mM.

12. The reagent system of claim 1, wherein the second surfactant is present in the second reagent at a concentration of 10 to 50 mM.

13. The reagent system of claim 1, wherein the base is present in the second reagent at a concentration of 25 to 150 mM, and
wherein the second surfactant comprises cetyltrimethylammonium chloride (CTAC) or cetyltrimethylammonium bromide (CTAB).

14. The reagent system of claim 1, wherein the base is present in the second reagent at a concentration of 100 to 400 mM, and
wherein the second surfactant comprises Triton X-100 or Tween 20.

15. The reagent system of claim 1, wherein the wash buffer further comprises a preservative.

16. The reagent system of claim 15, wherein the preservative comprises one or more selected from the group consisting of sodium azide, 5-bromo-5-nitro-1,3-dioxane, and a mixture of 5-chloro-2-methyl-4-isothiazolin-3-one with 2-methyl-4-isothiazolin-3-one.

17. A method of detecting a target antigen in a sample, comprising:
performing an immunoreaction between a target antigen in a sample and a detection antibody conjugated with acridinium ester for sandwich immunoassays or an artificial antigen, which is compatible with the target antigen, conjugated with acridinium ester for competitive immunoassays to form an acridinium ester-labeled immunocomplex;
mixing a wash buffer including a first surfactant in one or more of a phosphate-buffered saline (PBS) and a tris-buffered saline (TBS) with the acridinium ester-labeled immunocomplex to form micelles around the acridinium ester-labeled immunocomplex;
dispersing the acridinium ester-labeled immunocomplex within the micelles in a first reagent comprising hydrogen peroxide and an acid, and optionally a first alkali metal salt;
adding a second reagent including a base and a second surfactant, and optionally a second alkali metal salt, into the dispersed acridinium ester-labeled immunocomplex to emit a chemiluminescence signal; and
measuring an intensity of the chemiluminescence signal,
wherein the first surfactant and the second surfactant are independently one or more of a cationic surfactant, a neutral surfactant and an anionic surfactant,
wherein the acid has an acid dissociation constant (pKa) of −1.0 to 5.0, and
wherein the base comprises one or more of sodium hydroxide (NaOH), potassium hydroxide (KOH), ammonium hydroxide, tetra-n-ethylammonium hydroxide (TEA), tetra-n-propylammonium hydroxide (TPA), and tetra-n-butylammonium hydroxide (TBA).

18. The method of claim 17, wherein the dispersing step is performed in a reaction vessel, wherein the reaction vessel comprises the wash buffer in an amount of 50 μl or less and the acridinium ester-labeled immunocomplex.

19. The method of claim 17, wherein the cationic surfactant comprises cetyltrimethylammonium chloride (CTAC) or cetyltrimethylammonium bromide (CTAB).

20. The method of claim 17, wherein the neutral surfactant is one or more polyethylene glycol tert-octylphenyl ether derivatives and/or polyoxyethylenesorbitan monopalmitate derivatives selected from the group consisting of Triton X-45, Triton X-100,Triton X-114, Triton X-165, Triton X-305, Triton X-405, Triton X-705, Tween 20, Tween 40,Tween 60, and Tween 80.

21. The method of claim 17, wherein the anionic surfactant comprises ammonium dodecyl sulfate (ADS) or sodium dodecyl sulfate (SDS).

22. The method of claim 17, wherein the first surfactant is present in the wash buffer at a concentration of 0.05 to 0.3%.

23. The method of claim 17, wherein the graphene-based material is present in the first reagent at a concentration of 0.25 to 4 μg/ml.

24. The method of claim 17, wherein the hydrogen peroxide is present in the first reagent at a concentration of 2.5 to 40 mM, and
wherein the second surfactant comprises cetyltrimethylammonium chloride (CTAC) or cetyltrimethylammonium bromide (CTAB).

25. The method of claim 17, wherein the hydrogen peroxide is present in the first reagent at a concentration of 50 to 300 mM, and wherein the second surfactant comprises Triton X-100 or Tween 20.

26. The method of claim 17, wherein the acid is present in the first reagent at a concentration of 1 to 50 mM.

27. The method of claim 17, wherein the second surfactant is present in the second reagent at a concentration of 10 to 50 mM.

28. The method of claim 17, wherein the base is present in the second reagent at a concentration of 25 to 150 mM, and
wherein the second surfactant comprises cetyltrimethylammonium chloride (CTAC) or cetyltrimethylammonium bromide (CTAB).

29. The method of claim 17, wherein the base is present in the second reagent at a concentration of 100 to 400 mM, and
wherein the second surfactant comprises Triton X-100 or Tween 20.

30. The method of claim 17, wherein the wash buffer further comprises a preservative.

31. The method of claim 30, wherein the preservative comprises one or more selected from the group consisting of sodium azide, 5-bromo-5-nitro-1,3-dioxane, and a mixture of 5-chloro-2-methyl-4-isothiazolin-3-one with 2-methyl-4-isothiazolin-3-one.

32. The method of claim 17, wherein the sample is selected from the group consisting of serum, plasma, whole blood, stool, cerebrospinal fluid, synovial fluid, tissue, nasal swabs, and urine.

33. The method of claim 17, wherein the sample is selected from the group consisting of drinking water, tap water, a vegetable, a fruit, a meat, and a contaminated material.

34. The method of claim 17, wherein the target antigen is an antigen derived from bacteria, cells, foodborne pathogens, peptides, proteins, haptens, or viruses.

35. The method of claim 17, wherein the target antigen comprises a small molecule antigen, a non-protein antigen, a cyclic antigen or a single epitope antigen.

36. The method of claim 17, wherein the first reagent further comprises a graphene- based material selected from the group consisting of graphene, graphene oxide (GO), and reduced graphene oxide (rGO).
